SEQUENCE LISTING (2 sequences)

SEQ ID NO: 1 - protein, 1053 aa, Staphylococcus aureus
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 2 - DNA, 21 bp, Artificial Sequence
  Synthetic
gggtgagtgagtgtgtgcgtg

(19) United States
(12) Patent Application Publication
(10) Pub. No.: US 2023/0313159 A1
(43) Pub. Date: Oct. 5, 2023

(54) VARIANTS OF CAS NUCLEASE

(71) Applicant: ASC THERAPEUTICS, INC., Milpitas, CA (US)

(72) Inventors: H. Steve ZHANG, Moraga, CA (US); Qi ZHENG, San Ramon, CA (US)

(21) Appl. No.: 18/005,973

(22) PCT Filed: Jul. 26, 2021
(86) PCT No.: PCT/US2021/043098

§ 371 Date: Jan. 19, 2023

(30) Foreign Application Priority Data
Jul. 26, 2020 (US) 63056709

(51) Int. Cl. C12N 9/22

(52) U.S. Cl. CPC C12N 9/22

(57) ABSTRACT

Disclosed herein are variants of a Cas nuclease, polynucleotide encoding the same, compositions thereof, expression vectors, and methods of use thereof, for the generation of transgenic cells, tissues, plants, and animals. The compositions, vectors, and methods of the present invention are also useful in gene therapy and cell therapy techniques.

### CROSS-REFERENCE TO RELATED APPLICATIONS

This application claims priority to U.S. Provisional Application No. 63/056,709, filed Jul. 26, 2020, the disclosure of which is incorporated herein by reference.

### FIELD OF THE INVENTION

The present invention generally relates to compositions and methods used for genome engineering. More specifically, the present invention relates to Cas9 variants with improved specificity in genome engineering.

### BACKGROUND OF THE INVENTION

RNA-guided Cas nucleases derived from clustered regularly interspaced short palindromic repeats (CRISPR)-Cas systems have provided a versatile tool for editing the genome of diverse organisms. Specific cleavage of the intended nuclease target site without or with minimal off-target activity is a prerequisite for therapeutic applications of the CRISPR/Cas system. However, most Cas nucleases currently available exhibit significant off-target activity, and thus may not be suitable for clinical applications.

Among thousands of Cas proteins being discovered, the Staphylococcus aureus Cas9 (SaCas9) is especially important because of its relatively small size and high gene-editing efficiency. Still, the off-target issue is the main concern in its application, especially in therapeutics. Therefore, there remains a need for new compositions and methods for genome engineering technologies with improved specificity.

### BRIEF SUMMARY OF THE INVENTION

Disclosed herein are variants of a Cas nuclease, polynucleotide encoding the same, compositions thereof, expression vectors, and methods of use thereof, for genome engineering, the generation of transgenic cells, tissues, plants, and animals. The compositions, vectors, and methods of the present invention are also useful in gene therapy and cell therapy techniques.

In one aspect, the present disclosure provides a polypeptide comprising a variant of amino acid sequence of Staphylococcus aureus Cas9 (SaCas9), wherein the variant comprises at least 70% identity to SEQ ID NO: 1 and at least one mutation at an amino acid residue of SEQ ID NO: 1 which (a) is in the vicinity of gRNA nucleotide 12-14; (b) is in the bridge helix of SaCas9; or (c) forms a hydrogen bond with a target DNA.

In some embodiments, the variant has at least 75%, 80%, 85%, 90%, 95%, 96, %, 97%, 98%, 99% identity to SEQ ID NO: 1.

In some embodiments, the variant comprises at least one mutation is at the at an amino acid residue of SEQ ID NO: 1 selected from the group consisting of N44, R61, N120, T134, Y230, R245, K248, Y249, T316, S317, G391, T392, N413, N419, I445, L446, S447, K482, Y651, R654, D786, T787, Y789, K815, Y882, R1012, T1019 and S1022.

In some embodiments, the variant comprises at least one mutation at an amino acid residue of SEQ ID NO: 1 selected from the group consisting of N44, R61, K248, T316, S317, T392, N413, N419, K482 and R654.

In some embodiments, the mutation is selected from N44, R61, K248, T316, S317, T392 and K482.

In some embodiments, the mutation is selected from N44A, R61A, K248W, T316Y, S317Y, T392A and K482W.

In some embodiments, the mutation is selected from T316Y, S317Y and K482W.

In some embodiments, the mutation is N44A or R61A,

In some embodiments, the mutation is T392A or a combination of N413A, N419A and R654A.

In some embodiments, the mutation is (a) a combination of N44A and T316Y, or (b) a combination of R61A and T316Y, or (c) a combination of T316Y and T392A, or (d) a combination of T316Y and K482W, or (e) a combination of K482W and T392A, or (f) a combination of N413A, N419A, R654A and T316Y.

In another aspect, the present disclosure provides a polynucleotide encoding the polypeptide described herein.

In another aspect, the present disclosure provides a vector comprising the polynucleotide described herein. In some embodiments, the vector is a plasmid vector or a viral vector. In some embodiments, the vector is a lentiviral vector, a retroviral vector or an AAV vector.

In another aspect, the present disclosure provides a composition comprising the polypeptide described herein or a polynucleotide encoding the same, and a guide RNA. In some embodiments, the composition further comprises a donor DNA comprising a transgene.

In another aspect, the present disclosure provides a cell comprising a vector for expressing the polypeptide described herein.

In another aspect, the present disclosure provides a method for genome engineering in a cell comprising introducing the composition described herein into the cell. In some embodiments, the cell is a eukaryotic cell. In some embodiments, the cell is a mammalian or human cell. In some embodiments, the cell is a one-cell embryo.

These and other features, aspects, and advantages of the present invention will become better understood with regard to the following description, appended claims and accompanying drawings.

### BRIEF DESCRIPTION OF THE FIGURES

The following drawings form part of the present specification and are included to further demonstrate certain aspects of the present disclosure. The disclosure may be better understood by reference to one or more of these drawings in combination with the detailed description of specific embodiments presented herein.

FIG. 1 is a schematic illustration of position of guide RNA nucleotide

FIG. 2 is a schematic illustration of the crystal structure of SaCas9 which binds to the gRNA nucleotide. The nucleotides 12-14 are labeled in red.

FIG. 3 illustrates the amino acid sequence of wild type SaCas9 nuclease.

### DETAILED DESCRIPTION OF THE INVENTION

In the Summary of the Invention above and in the Detailed Description of the Invention, and the claims below, and in the accompanying drawings, reference is made to particular features (including method steps) of the invention. It is to be understood that the disclosure of the invention in this specification includes all possible combinations of such particular features. For example, where a particular feature is disclosed in the context of a particular aspect or embodiment of the invention, or particular claim, that feature can also be used, to the extent possible, in combination with and/or in the context of other particular aspects and embodiments of the invention, and in the invention generally.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

### Definition

As used herein, the singular forms “a”, “an” and “the” include plural references unless the context clearly dictates otherwise.

The term “Cas9” or “Cas9 nuclease” refers to an RNA-guided nuclease comprising a Cas9 protein, or a fragment thereof (e.g., a protein comprising an active or inactive DNA cleavage domain of Cas9, and/or the gRNA binding domain of Cas9). A Cas9 nuclease is also referred to sometimes as a casn1 nuclease or a CRISPR (clustered regularly interspaced short palindromic repeat)-associated nuclease. CRISPR is an adaptive immune system that provides protection against mobile genetic elements (viruses, transposable elements and conjugative plasmids). CRISPR clusters contain spacers, sequences complementary to antecedent mobile elements, and target invading nucleic acids. CRISPR clusters are transcribed and processed into CRISPR RNA (crRNA). In type II CRISPR systems correct processing of pre-crRNA requires a trans-encoded small RNA (tracrRNA), endogenous ribonuclease 3 (rnc) and a Cas9 protein. The tracrRNA serves as a guide for ribonuclease 3-aided processing of pre-crRNA. Subsequently, Cas9/crRNA/tracrRNA endonucleolytically cleaves linear or circular dsDNA target complementary to the spacer. The target strand not complementary to crRNA is first cut endonucleolytically, then trimmed 3′-5′ exonucleolytically. In nature, DNA-binding and cleavage typically requires protein and both crRNA and tracrRNA. The function of crRNA and tracrRNA can incorporated into a single guide RNA (“sgRNA”, or simply “gNRA”). See e.g., Jinek M., Chylinski K., Fonfara I., Hauer M., Doudna J. A., Charpentier E. Science 337:816-821 (2012), the entire contents of which is hereby incorporated by reference. Cas9 recognizes a short motif in the CRISPR repeat sequences (the PAM or protospacer adjacent motif) to help distinguish self versus non-self Cas9 nuclease sequences and structures are well known to those of skill in the art. Cas9 orthologs have been described in various species, including, but not limited to, S. pyogenes, S. mutans, S. thermophilus, C. jejuni, N. meningitides, P. multocida, F. novicida and S aureus.

It is noted that in this disclosure, terms such as “comprises”, “comprised”, “comprising”, “contains”, “containing” and the like are inclusive or open-ended and do not exclude additional, un-recited elements or method steps. Terms such as “consisting essentially of” and “consists essentially of” allow for the inclusion of additional ingredients or steps that do not materially affect the basic and novel characteristics of the claimed invention. The terms “consists of” and “consisting of” are close ended.

The term “effective amount,” as used herein, refers to an amount of a biologically active agent that is sufficient to elicit a desired biological response. For example, in some embodiments, an effective amount of a nuclease may refer to the amount of the nuclease that is sufficient to induce cleavage of a target site specifically bound and cleaved by the nuclease. As will be appreciated by the skilled artisan, the effective amount of an agent, e.g., a nuclease, may vary depending on various factors as, for example, on the desired biological response, the specific allele, genome, target site, cell, or tissue being targeted, and the agent being used.

The term “homologous,” as used herein is an art-understood term that refers to nucleic acids or polypeptides that are highly related at the level of nucleotide and/or amino acid sequence. Nucleic acids or polypeptides that are homologous to each other are termed “homologues.” Homology between two sequences can be determined by sequence alignment methods known to those of skill in the art. In accordance with the invention, two sequences are considered to be homologous if they are at least about 50-60% identical, e.g., share identical residues (e.g., amino acid residues) in at least about 50-60% of all residues comprised in one or the other sequence, at least about 70% identical, at least about 80% identical, at least about 90% identical, at least about 95% identical, at least about 98% identical, at least about 99% identical, at least about 99.5% identical, or at least about 99.9% identical, for at least one stretch of at least 20, at least 30, at least 40, at least 50, at least 60, at least 70, at least 80, at least 90, at least 100, at least 120, at least 150, or at least 200 amino acids.

The term “mutation,” as used herein, refers to a substitution of a residue within a sequence, e.g., a nucleic acid or amino acid sequence, with another residue, or a deletion or insertion of one or more residues within a sequence. Mutations are typically described herein by identifying the original residue followed by the position of the residue within the sequence and by the identity of the newly substituted residue. Various methods for making the amino acid substitutions (mutations) provided herein are well known in the art, and are provided by, for example, Green and Sambrook, Molecular Cloning: A Laboratory Manual (4^{th} ed., Cold Spring Harbor Laboratory Press, Cold Spring Harbor, N.Y. (2012)).

The term “nuclease,” as used herein, refers to an agent, for example, a protein, capable of cleaving a phosphodiester bond connecting two nucleotide residues in a nucleic acid molecule. In some embodiments, a nuclease is a protein, e.g., an enzyme that can bind a nucleic acid molecule and cleave a phosphodiester bond connecting nucleotide residues within the nucleic acid molecule. A nuclease may be an endonuclease, cleaving a phosphodiester bonds within a polynucleotide chain, or an exonuclease, cleaving a phosphodiester bond at the end of the polynucleotide chain. In some embodiments, a nuclease is a site-specific nuclease, binding and/or cleaving a specific phosphodiester bond within a specific nucleotide sequence, which is also referred to herein as the “recognition sequence,” the “nuclease target site,” or the “target site.” In some embodiments, a nuclease is an RNA-guided (i.e., RNA-programmable) nuclease, which is associated with (e.g., binds to) an RNA (e.g., a guide RNA, “gRNA”) having a sequence that complements a target site, thereby providing the sequence specificity of the nuclease. In some embodiments, a nuclease recognizes a single stranded target site, while in other embodiments, a nuclease recognizes a double-stranded target site, for example, a double-stranded DNA target site. The target sites of many naturally occurring nucleases, for example, many naturally occurring DNA restriction nucleases, are well known to those of skill in the art. In many cases, a DNA nuclease, such as EcoRI, HindIII, or BamHI, recognize a palindromic, double-stranded DNA target site of 4 to 10 base pairs in length, and cut each of the two DNA strands at a specific position within the target site. Some endonucleases cut a double-stranded nucleic acid target site symmetrically, i.e., cutting both strands at the same position so that the ends comprise base-paired nucleotides, also referred to herein as blunt ends. Other endonucleases cut a double-stranded nucleic acid target sites asymmetrically, i.e., cutting each strand at a different position so that the ends comprise unpaired nucleotides. Unpaired nucleotides at the end of a double-stranded DNA molecule are also referred to as “overhangs,” e.g., as “5′-overhang” or as “3′-overhang,” depending on whether the unpaired nucleotide(s) form(s) the 5′ or the 5′ end of the respective DNA strand. Double-stranded DNA molecule ends ending with unpaired nucleotide(s) are also referred to as sticky ends, as they can “stick to” other double-stranded DNA molecule ends comprising complementary unpaired nucleotide(s). A nuclease protein typically comprises a “binding domain” that mediates the interaction of the protein with the nucleic acid substrate, and also, in some cases, specifically binds to a target site, and a “cleavage domain” that catalyzes the cleavage of the phosphodiester bond within the nucleic acid backbone. In some embodiments a nuclease protein can bind and cleave a nucleic acid molecule in a monomeric form, while, in other embodiments, a nuclease protein has to dimerize or multimerize in order to cleave a target nucleic acid molecule.

The terms “nucleic acid molecule” and “polynucleotide” are used interchangeably and refer to a polymeric form of nucleotides of any length, either deoxyribonucleotides or ribonucleotides, or analogs thereof. Polynucleotides may have any three-dimensional structure, and may perform any function, known or unknown. Non-limiting examples of polynucleotides include a gene, a gene fragment, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers.

The term “pharmaceutical composition,” as used herein, refers to a composition that can be administrated to a subject in the context of treatment and/or prevention of a disease or disorder. In some embodiments, a pharmaceutical composition comprises an active ingredient, e.g., a nuclease or fragment thereof (or a nucleic acid encoding the same), and optionally a pharmaceutically acceptable excipient. In some embodiments, a pharmaceutical composition comprises inventive Cas9 variant protein(s) and gRNA(s) suitable for targeting the Cas9 variant to a target nucleic acid. In some embodiments, the target nucleic acid is a gene. In some embodiments, the target nucleic acid is an allele associated with a disease, whereby the allele is cleaved by the action of the Cas9 variant.

The terms “protein,” “peptide,” and “polypeptide” are used interchangeably herein, and refer to a polymer of amino acid residues linked together by peptide (amide) bonds. The terms refer to a protein, peptide, or polypeptide of any size, structure, or function. Typically, a protein, peptide, or polypeptide will be at least three amino acids long. A protein, peptide, or polypeptide may refer to an individual protein or a collection of proteins. One or more of the amino acids in a protein, peptide, or polypeptide may be modified, for example, by the addition of a chemical entity such as a carbohydrate group, a hydroxyl group, a phosphate group, a farnesyl group, an isofarnesyl group, a fatty acid group, a linker for conjugation, functionalization, or other modification, etc. A protein, peptide, or polypeptide may also be a single molecule or may be a multi-molecular complex. A protein, peptide, or polypeptide may be just a fragment of a naturally occurring protein or peptide. A protein, peptide, or polypeptide may be naturally occurring, recombinant, or synthetic, or any combination thereof. Any of the proteins provided herein may be produced by any method known in the art. For example, the proteins provided herein may be produced via recombinant protein expression and purification. Methods for recombinant protein expression and purification are well known, and include those described by Green and Sambrook, Molecular Cloning: A Laboratory Manual (4^{th} ed., Cold Spring Harbor Laboratory Press, Cold Spring Harbor, N.Y. (2012)), the entire contents of which are incorporated herein by reference.

The term “subject,” as used herein, refers to an individual organism, for example, an individual mammal. In some embodiments, the subject is a human. In some embodiments, the subject is a non-human mammal. In some embodiments, the subject is a non-human primate. In some embodiments, the subject is a rodent. In some embodiments, the subject is a sheep, a goat, a cattle, a cat, or a dog. In some embodiments, the subject is a vertebrate, an amphibian, a reptile, a fish, an insect, a fly, or a nematode. In some embodiments, the subject is a research animal. In some embodiments, the subject is genetically engineered, e.g., a genetically engineered non-human subject. The subject may be of either sex and at any stage of development.

The terms “target nucleic acid,” and “target genome,” as used herein in the context of nucleases, refer to a nucleic acid molecule or a genome, respectively, that comprises at least one target site of a given nuclease.

The term “target site” used herein refers to a sequence within a nucleic acid molecule that is bound and cleaved by a nuclease (e.g., Cas9 proteins provided herein). A target site may be single-stranded or double-stranded. In the context of a Cas9 nuclease, a target site typically comprises a nucleotide sequence that is complementary to the gRNA(s) of the Cas9 nuclease, and a protospacer adjacent motif (PAM) at the 3′ end adjacent to the gRNA-complementary sequence(s).

A “variant” of a polypeptide (e.g., a Cas9 nuclease) comprises an amino acid sequence wherein one or more amino acid residues are inserted into, deleted from and/or substituted into the amino acid sequence relative to another polypeptide sequence.

The term “vector” refers to a polynucleotide comprising one or more polynucleotides of the present invention, e.g., those encoding a Cas9 protein and/or gRNA provided herein. Vectors include, but are not limited to, plasmids, viral vectors, cosmids, artificial chromosomes, and phagemids. The vector is able to replicate in a host cell and is further characterized by one or more endonuclease restriction sites at which the vector may be cut and into which a desired nucleic acid sequence may be inserted. Vectors may contain one or more marker sequences suitable for use in the identification and/or selection of cells which have or have not been transformed or genomically modified with the vector. Markers include, for example, genes encoding proteins which increase or decrease either resistance or sensitivity to antibiotics (e.g., kanamycin, ampicillin) or other compounds, genes which encode enzymes whose activities are detectable by standard assays known in the art (e.g., (β-galactosidase, alkaline phosphatase, or luciferase), and genes which visibly affect the phenotype of transformed or transfected cells, hosts, colonies, or plaques. Any vector suitable for the transformation of a host cell (e.g., E. coli, mammalian cells such as CHO cell, insect cells, etc.) as embraced by the present invention, for example, vectors belonging to the pUC series, pGEM series, pET series, pBAD series, pTET series, or pGEX series. In some embodiments, the vector is suitable for transforming a host cell for recombinant protein production. Methods for selecting and engineering vectors and host cells for expressing proteins (e.g., those provided herein), transforming cells, and expressing/purifying recombinant proteins are well known in the art, and are provided by, for example, Green and Sambrook, Molecular Cloning: A Laboratory Manual (4^{th} ed., Cold Spring Harbor Laboratory Press, Cold Spring Harbor, N.Y. (2012)).

Polypeptides and SaCas9 Nuclease Variants

Site-specific nucleases are powerful tools for targeted genome modification in vitro and in vivo. Site-specific nuclease cleavage in living cells triggers a DNA repair mechanism that frequently results in a modification of the cleaved and repaired genomic sequence, for example, via homologous recombination. Accordingly, the targeted cleavage of a specific sequence within a genome opens up new avenues for gene targeting and gene modification in living cells, including cells that are hard to manipulate with conventional gene targeting methods, such as many human somatic or embryonic stem cells.

One concern of site-specific genomic modification is the possibility of off-target nuclease effects, e.g., the cleavage of genomic sequences that differ from the intended target sequence by one or more nucleotides. Undesired side effects of off-target cleavage range from insertion into unwanted loci during a gene targeting event to severe complications in a clinical scenario. Off-target cleavage of sequences encoding essential gene functions or tumor suppressor genes by an endonuclease administered to a subject may result in disease or even death of the subject. Accordingly, it is desirable to design and develop new nucleases having the greatest chance of minimizing off-target effects.

The methods and compositions of the present disclosure represent, in some aspects, an improvement over previous methods and compositions providing nucleases (and methods of their use) engineered to have improved specificity for their intended targets. Accordingly, aspects of the present disclosure aim at reducing the chances for Cas9 off-target effects using novel engineered Cas9 variants. In one example, a Cas9 variant is provided which has improved specificity as compared to the wild type Cas9, exhibiting, e.g., >2-fold, >5-fold, >10-fold, >50-fold, >100-fold, >140-fold, >200-fold, or more, higher specificity than a wild type Cas9.

In one aspect, the present disclosure provides a Cas9 variant based on Staphylococcus aureus Cas9 (SaCas9) nuclease. SaCas9 has its importance in genome engineering application because of its smaller size (1053 amino acid residues) compared to other Cas9 nuclease, e.g., SpCas9. SaCas9 recognizes an NNGRRT protospacer adjacent motif (PAM). Typically, the SaCas9 nuclease employs a 21 nucleotides gRNA to guide the nuclease binding to its target DNA. In some embodiments, the amino acid sequence of a wild-type SaCas9 nuclease is illustrated in SEQ ID NO: 1.

In some embodiments, the SaCas9 nuclease variant provided herein has at least 70% identity to SEQ ID NO: 1 and at least one mutation at an amino acid residue of SEQ ID NO: 1 selected from the group consisting of N44, R61, N120, T134, Y230, R245, K248, Y249, T316, S317, G391, T392, N413, N419, I445, L446, S447, K482, Y651, R654, D786, T787, Y789, K815, Y882, R1012, T1019 and S1022.

In the context of a nuclease variant, the term “percentage identity” and “% identity” between two amino acid (peptide) or nucleic acid (nucleotide) sequences means the percentage of identical amino acid or nucleotide residues in corresponding positions in the two optimally aligned sequences.

To determine the “percentage identity” of the two amino acid or nucleic acid sequences, the sequences are aligned together. To achieve an optimal match, gaps can be introduced into the sequence (i.e. deletions or insertions which can also be placed at the sequence ends). Amino acid and nucleotide residues in the corresponding positions are then compared. When a position in the first sequence is occupied by the same amino acid or nucleotide residue that occupies the corresponding position in the second sequence, the molecules are identical in that position. The percentage identity between two sequences is a function of the number of identical positions divided by the sequences, i.e.,

% identity=(number of identical positions/total number of positions)×100

According to an advantageous embodiment, the sequences have the same length. Advantageously, the compared sequences do not have gaps (or insertions).

The percentage identity can be obtained by using mathematical algorithms. A non-limiting example of an algorithm used for comparing two sequences is the Karlin and Altschul algorithm (Proc. Natl. Acad. Sci. USA 87 (1990) 2264-2268) modified by Karlin and Altschul (Proc. Natl. Acad. Sci. USA 90 (1993) 5873-5877]. Said algorithm is incorporated in the BLASTn and BLASTp programs of Altschul (Altschul et al, J. Mol. Bio. 215 (1990) 403-410).

With the purpose of achieving alignments even in the presence of one or more gaps (or insertions) methods may be used which assign a relatively high penalty for each gap (or insertion) and a lower penalty for each additional amino acid or nucleotide residue in the gap (this additional amino acid or nucleotide residue is defined as gap extension). High penalties will obviously lead to the alignments being optimized with the least number of gaps.

An example of a program able to achieve this type of alignment is the BLAST program as described in Altschul et al., Nucleic Acids Res. 25 (1997) 3389-3402. For this purpose the BLASTn and BLASTp programs can be used with the default parameters. When using the BLAST program, the BLOSUM62 matrix is typically employed.

An advantageous and non-limiting example of a program for achieving an optimal alignment is GCG Wisconsin Bestfit package (University of Wisconsin, USA; Devereux et. al., 1984, Nucleic Acid Research 12:387). The default parameters are again used i.e. for an amino acid sequence they allow a penalty of −12 for a gap and a penalty of −4 for each extension.

In some embodiments, the SaCas9 nuclease variant provided herein has at least one mutation at an amino acid residue of the wide-type SaCas9 protein that is in the vicinity of gRNA nucleotide 12-14. In some embodiments, the amino acid residue of the wild-type SaCas9 protein that is in the vicinity of gRNA nucleotide 12-14 is selected from I445, L446, S447, Y651, T316, S317, K248, Y249, and K482. In some embodiments, the amino acid residue of the wild type SaCas9 protein that is in the vicinity of gRNA nucleotide 12-14 is T316, S317, K482 and K248.

In some embodiments, the mutation involves a substitution of the wide type amino acid residue with an amino acid residue having a larger side chain. In some embodiments, the amino acid residue used for substitution is a tyrosine (Y), tryptophan (W), leucine (L), isoleucine (I), asparagine (N) or glutamine (Q). In some embodiments, the substitution is selected from T316Y, S317Y, K248W, and K482W.

In some embodiments, the SaCas9 nuclease variant provided herein has at least one mutation at an amino acid residue in the bridge helix of a wide-type SaCas9 protein. In some embodiments, the amino acid residue in the bridge helix of a wide-type SaCas9 protein forms a hydrogen bond with the gRNA. In some embodiments, the mutation at the amino acid residue in the bridge helix abolishes the hydrogen bond with the gRNA. In some embodiments, the amino acid residues in the bridge helix is N44 or R61. In some embodiments, mutation is a substitution with an amino acid residue selected from alanine (A), glycine (G) or valine (V). In some embodiments, the mutation is N44A or R61A.

In some embodiments, SaCas9 nuclease variant provided herein has at least one mutation at an amino acid residue of a wide-type SaCas9 protein that forms a hydrogen bond with a target DNA. In some embodiments, the amino acid residue of a wide-type SaCas9 protein that forms a hydrogen bond with a target DNA is selected from N120, T134, Y230, R245, G391, T392, N413, N419, R654, D786, T787, Y789, K815, Y882, R1012, T1019 and S1022. In some embodiments the mutation is a substitution with an amino acid residue selected from alanine (A), glycine (G) or valine (V). In some embodiments, the mutation is T392A or a combination of N413A/N419A/R654A.

In some embodiments, the SaCas9 variant provided herein has one or more conservative substitutions of the amino acids in SEQ ID NO: 1. In this context, a conservative substitution means that the resulting variant does not substantially alter the biological activity of a SaCas9 nuclease. Suitable conservative substitutions of amino acids are known to those of skill in this art. In general, single amino acid substitutions in non-essential regions of a polypeptide do not substantially alter biological activity (see, e.g. Watson et al. Molecular Biology of the Gene, 4th Edition, 1987, Benjamin/Cummings, p. 224). In particular, such a conservative variant has a modified amino acid sequence, such that the change(s) do not substantially alter the protein's (the conservative variant's) structure and/or activity, e.g., enzymatic activity. These include conservatively modified variations of an amino acid sequence, i.e., amino acid substitutions, additions or deletions of those residues that are not critical for protein activity, or substitution of amino acids with residues having similar properties (e.g., acidic, basic, positively or negatively charged, polar or non-polar, etc.) such that the substitutions of even critical amino acids does not substantially alter structure and/or activity. Conservative substitution tables providing functionally similar amino acids are well known in the art. For example, one exemplary guideline to select conservative substitutions includes (original residue followed by exemplary substitution): Ala/Gly or Ser; Arg/Lys; Asn/Gln or His; Asp/Glu; Cys/Ser; Gln/Asn; Gly/Asp; Gly/Ala or Pro; His/Asn or Gln; Ile/Leu or Val; Leu/Ile or Val; Lys/Arg or Gln or Glu; Met/Leu or Tyr or He; Phe/Met or Leu or Tyr; Ser/Thr; Thr/Ser; Trp/Tyr; Tyr/Trp or Phe; Val/Ile or Leu. An alternative exemplary guideline uses the following six groups, each containing amino acids that are conservative substitutions for one another: (1) alanine (A or Ala), serine (S or Ser), threonine (T or Thr); (2) aspartic acid (D or Asp), glutamic acid (E or Glu); (3) asparagine (N or Asn), glutamine (Q or Gln); (4) arginine (R or Arg), lysine (K or Lys); (5) isoleucine (I or He), leucine (L or Leu), methionine (M or Met), valine (V or Val); and (6) phenylalanine (F or Phe), tyrosine (Y or Tyr), tryptophan (W or Trp); (see also, e.g., Creighton (1984) Proteins, W. H. Freeman and Company; Schulz and Schimer (1979) Principles of Protein Structure, Springer-Verlag). One of skill in the art will appreciate that the above-identified substitutions are not the only possible conservative substitutions. For example, for some purposes, one may regard all charged amino acids as conservative substitutions for each other whether they are positive or negative. In addition, individual substitutions, deletions or additions that alter, add or delete a single amino acid or a small percentage of amino acids in an encoded sequence can also be considered “conservatively modified variations” when the three-dimensional structure and the function of the protein to be delivered are conserved by such a variation.

It can be understood that the SaCas9 nuclease variant described herein can be linked to a peptide or polypeptide at either N or C terminus. Thus, in another aspect, the present disclosure provides a polypeptide that contains any one of the SaCas9 nuclease variants described herein and one or more (poly)peptides linked to the SaCas9 variant. The examples of the (poly)peptides that can be linked to the SaCas9 variant include, without limitation, a tag (e.g., 6×HIS tag, HA tag, etc.), a nuclear localization signal (NLS) domain, a recombinase, a transposase, etc.

Polynucleotides, Vectors, Cells, Kits

In another aspect, the present disclosure provides polynucleotides encoding one or more of the inventive proteins described herein. In some embodiments, the polynucleotides are provided for expressing the SaCas9 nuclease variants described herein. In some embodiments, the polynucleotide is for expressing the SaCas9 nuclease variant in a cell for genome engineering of the cell. In some embodiments, the polynucleotides are provided for recombinant expression and purification of SaCas9 nuclease variants described herein. In some embodiments, the polynucleotide comprises a sequence encoding any of the SaCas9 nuclease variants described herein and one or more sequences encoding a gRNA.

In general, a “CRISPR-Cas guide RNA” or “guide RNA” or gRNA refers to an RNA that directs sequence-specific binding of a CRISPR complex to the target sequence. In the context of a Cas9 nuclease, a typical guide RNA comprises (i) a guide sequence that has sufficient complementarity with a target polynucleotide sequence to hybridize with the target sequence and (ii) a trans-activating cr (tracr) mate sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), ClustalW, Clustal X, BLAT, Novoalign (Novocraft Technologies, ELAND (Illumina, San Diego, Calif.), SOAP (available at soap.genomics.org.cn), and Maq (available at maq.sourceforge.net). In some embodiments, a guide sequence is about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, a guide sequence is less than about 75, 50, 45, 40, 35, 30, 25, 20, 15, 12, or fewer nucleotides in length. The ability of a guide sequence to direct sequence-specific binding of a CRISPR complex to a target sequence may be assessed by any suitable assay known in the art. For example, the components of a CRISPR system sufficient to form a CRISPR complex, including the guide sequence to be tested, may be provided to a host cell having the corresponding target sequence, such as by transfection with vectors encoding the components of the CRISPR sequence, followed by an assessment of preferential cleavage within the target sequence. Similarly, cleavage of a target polynucleotide sequence may be evaluated in a test tube by providing the target sequence, components of a CRISPR complex, including the guide sequence to be tested and a control guide sequence different from the test guide sequence, and comparing binding or rate of cleavage at the target sequence between the test and control guide sequence reactions.

In another aspect, the present disclosure provides vectors comprising one or more polynucleotides encoding any of the SaCas9 nuclease variants described herein. In some embodiments, the vectors described herein is used for genome engineering in a cell. In some embodiments, the vectors described herein is used for recombinant expression and purification of SaCas9 nuclease variants. Typically, the vector comprises a sequence encoding a SaCas9 nuclease variant operably linked to a promoter, such that the SaCas9 nuclease variant is expressed in a host cell. In some embodiments, the vector comprises one or more sequences encoding a SaCas9 variant described herein, and a gRNA. In some embodiments, the vector further comprises a donor sequence or transgene to be inserted at the target site.

In another aspect, the present disclosure provides cells comprising a polynucleotide described herein. In some embodiments, the cell is for recombinant expression and purification of any of the SaCas9 nuclease variant provided herein. The cells include any cell suitable for recombinant protein expression, for example, cells comprising a genetic construct expressing or capable of expressing a SaCas9 nuclease variant described herein (e.g., cells that have been transformed with one or more vectors described herein, or cells having genomic modifications, for example, those that express a protein provided herein from an allele that has been incorporated in the cell's genome). Methods for transforming cells, genetically modifying cells, and expressing genes and proteins in such cells are well known in the art, and include those provided by, for example, Green and Sambrook, Molecular Cloning: A Laboratory Manual (4^{th} ed., Cold Spring Harbor Laboratory Press, Cold Spring Harbor, N.Y. (2012)) and Friedman and Rossi, Gene Transfer: Delivery and Expression of DNA and RNA, A Laboratory Manual (1^{st} ed., Cold Spring Harbor Laboratory Press, Cold Spring Harbor, N.Y. (2006)).

In another aspect, the present disclosure provides kits comprising a SaCas9 nuclease variant as provided herein or a polynucleotide encoding the same. In some embodiments, the kit comprises a vector for expressing the SaCas9 nuclease variant described herein, wherein the vector comprises a polynucleotide encoding any of the SaCas9 nuclease variants provided herein. In some embodiments, the kit comprises a cell (e.g., any cell suitable for expressing a SaCas9 nuclease variant, such as bacterial, yeast, or mammalian cells) that comprises a genetic construct for expressing any of the SaCas9 nuclease variants provided herein. In some embodiments, any of the kits provided herein further comprise one or more gRNAs and/or vectors for expressing one or more gRNAs. In some embodiments, the kit comprises an excipient and instructions for contacting the nuclease with the excipient to generate a composition suitable for contacting a nucleic acid with the nuclease such that hybridization to and cleavage of a target nucleic acid occurs. In some embodiments, the composition is suitable for delivering a SaCas9 nuclease variant to a cell. In some embodiments, the composition is suitable for delivering a SaCas9 nuclease variant to a subject. In some embodiments, the excipient is a pharmaceutically acceptable excipient.

Methods of Genome Engineering

In another aspect, the present disclosure provides methods for genome engineering in a cell. In some embodiments, the method comprises introducing an effective amount of the SaCas9 nuclease variant described herein into the cell. In some embodiments, the SaCas9 nuclease variant is introduced into the cell by contacting the SaCas9 variant protein with the cell. In some embodiments, the SaCas9 nuclease variant is introduced into the cell by introducing a vector into the cell, wherein the vector comprises a polynucleotide encoding the SaCas9 variant.

Conventional viral and non-viral based gene transfer methods can be used to introduce the vectors in mammalian cells, target tissues or one-cell embryos. Such methods can be used to administer nucleic acids encoding components of the composition to cells in culture, or in a host organism. Non-viral vector delivery systems include DNA plasmids, RNA (e.g. a transcript of a vector described herein), naked nucleic acid, and nucleic acid complexed with a delivery vehicle, such as a liposome, protein complexed with a delivery vehicle, such as a liposome. Viral vector delivery systems include DNA and RNA viruses, which have either episomal or integrated genomes after delivery to the cell. For a review of gene therapy procedures, see Anderson, Science 256:808-813 (1992); Nabel & Felgner, TIBTECH 11:211-217 (1993); Mitani & Caskey, TIBTECH 11:162-166 (1993); Dillon, TIBTECH 11:167-175 (1993); Miller, Nature 357:455-460 (1992); Van Brunt, Biotechnology 6(10):1149-1154 (1988); Vigne, Restorative Neurology and Neuroscience 8:35-36 (1995); Kremer & Perricaudet, British Medical Bulletin 51(1):31-44 (1995); Haddada et al., in Current Topics in Microbiology and Immunology Doerfler and Bihm (eds) (1995); and Yu et al., Gene Therapy 1:13-26 (1994).

Methods of non-viral delivery of nucleic acids include lipofection, nucleofection, electroporation, microinjection, biolistics, virosomes, liposomes, immunoliposomes, polycation or lipid:nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Feigner, WO 91/17424; WO 91/16024. Delivery can be to cells (e.g. in vitro or ex vivo administration) or target tissues (e.g. in vivo administration).

The preparation of lipid:nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787).

Microinjection is used to deliver DNA, RNA or peptides into a nucleus and cytoplasm of a one-cell embryo. It is well known to one of skill in the art (see Manipulating the mouse embryo; A laboratory manual, fourth edition, 2014).

The use of RNA or DNA viral based systems for the delivery of nucleic acids take advantage of highly evolved processes for targeting a virus to specific cells in the body and trafficking the viral payload to the nucleus. Viral vectors can be administered directly to patients (in vivo) or they can be used to treat cells in vitro, and the modified cells may optionally be administered to patients (in vivo). Conventional viral based systems could include retroviral, lentiviral, adenoviral, adeno-associated and herpes simplex viral vectors for gene transfer. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues.

In some embodiments, the genome engineering via the method described herein involves a site-specific nucleic acid (e.g., DNA) cleavage. In some embodiments, the site-specific nucleic acid cleavage involves contacting a DNA with any of the SaCas9 nuclease variant described herein mediated by a guide RNA. For example, in some embodiments, the method comprises contacting a DNA with a SaCas9 nuclease variant, wherein the SaCas9 nuclease variant binds a gRNA that hybridizes to a region of the DNA. In some embodiments, the method has an on-target:off-target cleavage ratio that is at least 2-fold, at least 5-fold, at least 10-fold, at least 20-fold, at least 30-fold, at least 40-fold, at least 50-fold, at least 60-fold, at least 70-fold, at least 80-fold, at least 90-fold, at least 100-fold, at least 110-fold, at least 120-fold, at least 130-fold, at least 140-fold, at least 150-fold, at least 175-fold, at least 200-fold, or at least 250-fold or more higher than the on-target:off-target cleavage ratio of methods utilizing a wild type SaCas9 nuclease. Methods for determining on-target:off-target cleavage ratios are known, and include those described in the Examples.

In some embodiments, the site-specific nucleic acid cleavage involved in the method disclosed herein is followed by the modification of the nucleic acid, for example, a deletion, an insertion, an inversion, or a translocation.

In some embodiments, the genome engineering method provided herein further involves a recombination of two or more nucleic acids so as to insert a nucleic acid sequence into a target nucleic acid. In some embodiments, the genome engineering method further comprises into the cell a donor sequence to be inserted at the target site. In some embodiments, the donor sequence comprises a transgene. In some embodiments, the donor sequence is homologous to a genomic sequence at the target site, e.g., 1%, 2%, 3%, 4%, 5%, 10%, 20%, 30%, 40%, 50%, 60%, 70%, 80%, 85%, 90%, 95%, or 100% homologous to the nucleotide sequences flanking the target site, e.g., within about 100 bases or less of the target site, e.g. within about 90 bases, within about 80 bases, within about 70 bases, within about 60 bases, within about 50 bases, within about 40 bases, within about 30 bases, within about 15 bases, within about 10 bases, within about 5 bases, or immediately flanking the target site. In some embodiments, the donor sequence does not share any homology with the target nucleic acid, e.g., does not share homology to a genomic sequence at the target site. Donor sequences can be of any length, e.g., 10 nucleotides or more, 50 nucleotides or more, 100 nucleotides or more, 250 nucleotides or more, 500 nucleotides or more, 1000 nucleotides or more, 5000 nucleotides or more, 10000 nucleotides or more, 100000 nucleotides or more, etc.

Typically, the donor sequence is not identical to the target sequence that it replaces or is inserted into. In some embodiments, the donor sequence contains at least one or more single base changes, insertions, deletions, inversions or rearrangements with respect to the target sequence (e.g., target genomic sequence). In some embodiments, donor sequences also comprise a vector backbone containing sequences that are not homologous to the DNA region of interest and that are not intended for insertion into the DNA region of interest.

The donor sequence may comprise certain sequence differences as compared to the target (e.g., genomic) sequence, for example restriction sites, nucleotide polymorphisms, selectable markers (e.g., drug resistance genes, fluorescent proteins, enzymes etc.), which can be used to assess for successful insertion of the donor sequence at the target site or in some cases may be used for other purposes (e.g., to signify expression at the targeted genomic locus). In some embodiments, if located in a coding region, such nucleotide sequence differences will not change the amino acid sequence, or will make silent amino acid changes (e.g., changes which do not affect the structure or function of the protein).

The donor sequence may be provided to the cell as single-stranded DNA, single-stranded RNA, double-stranded DNA, or double-stranded RNA. It may be introduced into a cell in linear or circular form. If introduced in linear form, the ends of the donor sequence may be protected (e.g., from exonucleolytic degradation) by methods known to those of skill in the art. For example, one or more dideoxynucleotide residues are added to the 3′ terminus of a linear molecule and/or self-complementary oligonucleotides are ligated to one or both ends. See, e.g., Chang et al., Proc. Natl. Acad Sci USA. 1987; 84:4959-4963; Nehls et al., Science. 1996; 272:886-889. In some embodiments, a donor sequence can be introduced into a cell as part of a vector molecule having additional sequences such as, for example, replication origins, promoters and genes encoding antibiotic resistance. In some embodiments, donor sequences can be introduced as naked nucleic acid, as nucleic acid complexed with an agent such as a liposome or poloxamer, or can be delivered by viruses (e.g., adenovirus, AAV, etc.).

In some embodiments, genome engineering method described herein is performed in a cell, for example, a bacterium, a yeast cell, or a mammalian cell. In some embodiments, genome engineering method provided herein is performed in a eukaryotic cell. In some embodiments, the genome engineering method is performed in a cell or tissue in vitro or ex vivo. In some embodiments, the genome engineering method is performed in an individual, such as a patient or research animal. In some embodiment, the individual is a human.

Pharmaceutical Composition

In another aspect, the present disclosure provides a pharmaceutical composition comprising any of the SaCas9 nuclease variants described herein. For example, some embodiments provide pharmaceutical compositions comprising a SaCas9 nuclease variant as provided herein, or a nucleic acid encoding such a variant, and a pharmaceutically acceptable excipient. Pharmaceutical compositions may optionally comprise one or more additional therapeutically active substances.

In some embodiments, compositions provided herein are administered to a subject, for example, to a human subject, in order to effect a targeted genomic modification within the subject. In some embodiments, cells are obtained from the subject and are contacted with a SaCas9 nuclease variant ex vivo. In some embodiments, cells removed from a subject and contacted ex vivo with an inventive nuclease variant are re-introduced into the subject, optionally after the desired genomic modification has been effected or detected in the cells. Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and other primates, mammals, domesticated animals, pets, and commercially relevant mammals such as cattle, pigs, horses, sheep, cats, dogs, mice, rats, and birds, including commercially relevant birds such as chickens, ducks, geese, and turkeys.

Formulations of the pharmaceutical compositions described herein may be prepared by any method known or hereafter developed in the art of pharmacology. In general, such preparatory methods include the step of bringing the active ingredient into association with an excipient, and then, if necessary or desirable, shaping and packaging the product into a desired single- or multi-dose unit.

Pharmaceutical formulations may additionally comprise a pharmaceutically acceptable excipient, which, as used herein, includes any and all solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired. Remington's The Science and Practice of Pharmacy, 21^{st} Edition, A. R. Gennaro (Lippincott, Williams & Wilkins, Baltimore, Md., 2006; incorporated in its entirety herein by reference) discloses various excipients used in formulating pharmaceutical compositions and known techniques for the preparation thereof. Except insofar as any conventional excipient medium is incompatible with a substance or its derivatives, such as by producing any undesirable biological effect or otherwise interacting in a deleterious manner with any other component(s) of the pharmaceutical composition, its use is contemplated to be within the scope of this disclosure.

In some embodiments, compositions in accordance with the present invention may be used for treatment of any of a variety of diseases, disorders, and/or conditions, including but not limited to one or more of the following: autoimmune disorders (e.g. diabetes, lupus, multiple sclerosis, psoriasis, rheumatoid arthritis); inflammatory disorders (e.g. arthritis, pelvic inflammatory disease); infectious diseases (e.g. viral infections (e.g., HIV, HCV, RSV), bacterial infections, fungal infections, sepsis); neurological disorders (e.g. Alzheimer's disease, Huntington's disease; autism; Duchenne muscular dystrophy); cardiovascular disorders (e.g. atherosclerosis, hypercholesterolemia, thrombosis, clotting disorders, angiogenic disorders such as macular degeneration); proliferative disorders (e.g. cancer, benign neoplasms); respiratory disorders (e.g. chronic obstructive pulmonary disease); digestive disorders (e.g. inflammatory bowel disease, ulcers); musculoskeletal disorders (e.g. fibromyalgia, arthritis); endocrine, metabolic, and nutritional disorders (e.g. diabetes, osteoporosis); urological disorders (e.g. renal disease); psychological disorders (e.g. depression, schizophrenia); skin disorders (e.g. wounds, eczema); blood and lymphatic disorders (e.g. anemia, hemophilia); etc.

The function and advantage of these and other embodiments of the present invention will be more fully understood from the Examples below. The following Examples are intended to illustrate the benefits of the present invention and to describe particular embodiments, but are not intended to exemplify the full scope of the invention. Accordingly, it will be understood that the Examples are not meant to limit the scope of the invention.

### Example 1

This example illustrates the generation of SaCas9 nuclease variant that has improved specificity, wherein the variant has a mutation that blocks the “r12-14 opening.”

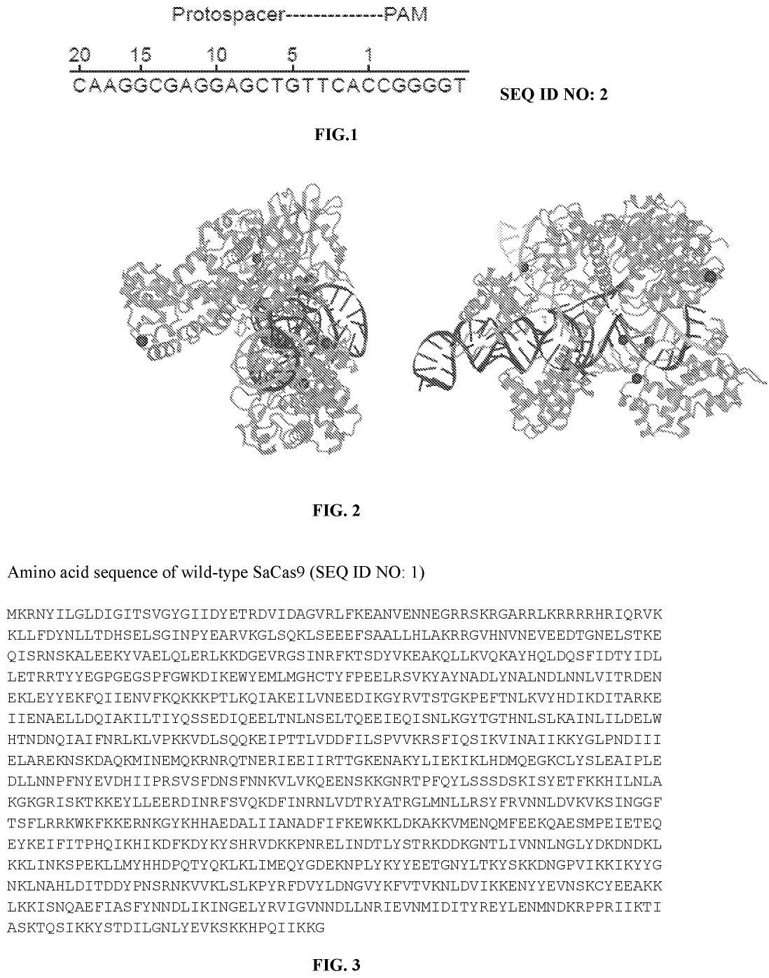

Typically, SaCas9 nuclease employs a 21nt guide RNA (gRNA) to guide the nuclease binding to its target DNA. Through datamining the published research articles, the inventors noted that, compare to the on-target site, most of the off-target sites contain mismatched bases between position 12 and 14 (position 1 being the 1^{st} nucleotide 5′ to the PAM sequence NNGRRT, FIG. 1). According to the crystal structure, this segment of the DNA/RNA complex faces an opening of SaCas9 (FIG. 2). Because non-complimentary DNA/RNA bases result in a less compact structure relative to complimentary ones, reducing the size of the opening on SasCas9 may improve its specificity. The inventors analyzed the SaCas9 structure to identify amino acids residues that are in the vicinity of gRNA nucleotide 12-14. Substituting these residues with ones with a larger side chain can improve the enzyme specificity. These residues include I445, L446, S447, Y651, T316, S317, K248, Y249, K482. The best candidates are T316, S317, K482 and K248, for example, T316Y, S317Y, and K482W.

VEGFA_8 gRNA (GGGTGAGTGAGTGTGTGCGTG, SEQ ID NO: 2), a published gRNA with well documented off-target sites, was selected to evaluate the specificity of different SaCas9 variants. A 34-bp double-stranded oligodeoxynucleotide (dsODN) was co-transfected with plasmids encoding SaCas9 variants and the gRNA plasmid. Modification of the top off-target sites were analyzed using deep sequencing. The result is listed in Table 1, these variants showed comparable on-target efficiency (63-84% of wild type saCas9) and less off-target cutting.

TABLE 1
Deep-sequencing analysis of SaCas9 variants T316Y, S317Y and K482W for on-target and off-target of
VEGFA_8 gRNA (the % of indel and % of dsODN integration at each site are listed, % indel/% dsODN integration)

| SaCas9 | Wt-1 | Wt-2 | Wt-3 | Wt-4 | K248W | T316Y-1 | T316Y-2 | S317Y | K482W |
|---|---|---|---|---|---|---|---|---|---|
| On-target | 70.4/13.84 | 46.3/12.13 | 66.8/19.44 | 72.54/3.59 | 51.01/3.76 | 59.1/13.48 | 65.62/3.67 | 54.3/9.48 | 42.2/23.78 |
| OT1 | 0.05/0 | 13.0/4.23 | 0.06/0.06 | 0.06/0 | 0/0 | 0/0 | 0/0 | 0/0 | 0/0 |
| OT2 | — | — | — | 0.16/0 | 0/0 | | 0/0 | | |
| OT3 | 0/0 | 0.64/0.08 | 0.21/0.06 | 0.12/0 | 0/0 | 0/0 | 0/0 | 0/0 | 0/0 |
| OT4 | 0/0 | 0.5/0.11 | 0/0 | 0/0 | 0/0 | 0/0 | 0/0 | 0/0 | 0/0 |
| OT7 | 0/0 | — | 0.23/0 | 0/0 | 0/0 | 0/0 | 0/0 | 0/0 | 0/0 |
| OT10 | 0/0 | 0/0 | 0/0 | 0/0 | 0/0 | 0/0 | 0/0 | 0/0 | |
| OT13 | 0/0 | 0.07/0.07 | 0/0 | 0.00/0 | 0.00/0 | 0/0 | 0/0 | 0/0 | 0/0 |
| OT16 | 0/0 | 0/0 | 0/0 | 0/0 | 0/0 | 0/0 | 0/0 | 0/0 | 0/0 |
| OT17 | 0/0 | 0/0 | 0/0 | 0/0 | 0/0 | 0/0 | 0/0 | 0/0 | 0/0 |
| OT18 | — | — | 0/0 | 1.2/0 | 0/0 | — | 0/0 | — | 0/0 |
| OT21 | 0.79/0.27 | 0/0 | 0.23/0.15 | 0.19/0 | 0/0 | 0/0 | 0/0 | 0.45/0.25 | 0/0 |

### Example 2

This example illustrates the generation of SaCas9 nuclease variant that has improved specificity, wherein the variant has a mutation of bridge helix

Bridge helix is essential for initiation and stability of R-loop. Mutation of the arginine residues in bridge helix was confirmed to affect the on-target and off-target activity dramatically in Streptococcus pyogenes Cas9 (SpCas9) (Bratovic M (2020) Nature Chemical Biology 16: 587-592). The inventors identified all hydrogen bonds between the bridge helix and gRNA, made individual amino acid substitutions to remove each hydrogen bond one by one. This approach results in two SaCas9 variants with higher specificity, N44A and R61A.

The evaluation process is same as in Example 1. As shown in Table 2, both N44A and R61A showed similar on-target activity and lower off-target editing compared to the wild-type SaCas9.

TABLE 2
Deep-sequencing analysis of SaCas9 variants N44A and
R61A for on-target and off-target of VEGFA_8 gRNA.
(The % of indel and % of dsODN integration at each
site are listed, % indel/% dsODN integration)

| SaCas9 | wt | N44A | R61A |
|---|---|---|---|
| On-target | 64.6/14.57 | 50.5/11.82 | 40.7/10.44 |
| OT1 | 0.06/0.06 | 0/0 | 0/0 |
| OT3 | 0.28/0.19 | 0/0 | 0/0 |
| OT4 | 0/0 | 0/0 | 0/0 |
| OT7 | 0/0 | 0/0 | 0/0 |
| OT10 | 0/0 | 0/0 | 0/0 |
| OT13 | 0/0 | 0/0 | 0/0 |
| OT16 | 0/0 | 0/0 | 0/0 |
| OT17 | 0/0 | 0/0 | 0/0 |
| OT20 | 0/0 | 0/0 | 0/0 |
| OT21 | 0.44/0.22 | 0.44/0.33 | 0/0 |

### Example 3

This example illustrates the generation of SaCas9 nuclease variant with mutations that has improved specificity, wherein the variant has a mutation that removes the hydrogen bonds between SaCas9 and the target DNA.

This strategy was originally confirmed by J. Keith Joung group using SpCas9 (Kleinstiver BP (2016) Nature 529: 490-495). This method focuses on the hydrogen bonds between the nuclease and its target DNA. Jiahai Shi and Zongli Zheng group in City University of Hong Kong tested four residues in SaCas9 and found that a quadruple substitution variant (N413A/N419A/R245A/R654A), which was called SaCas9-HF, showed higher specificity (Tan Y (2019) PNAS 116: 20969-20976). However, the published data has its limitations. First, the hydrogen bonds are target DNA-specific; the amino acid substitutions described in SaCas9-HF may have different effects on other target DNA sequences. Second, the four residues in publication did not cover all hydrogen bonds between SaCas9 and target DNA in its crystal structure. Third, the SaCas9-HF showed relatively low on-target activity. The inventors evaluated all residues showing hydrogen bond to target DNA in the SaCas9 crystal structure, including N120, T134, Y230, R245, G391, T392, N413, N419, R654, D786, T787, Y789, K815, Y882, R1012, T1019 and S1022. Among this list, T392A showed a better specificity compared to wild type SaCas9 (see Table 3). The triple mutation N413A/N419A/R654A also showed improved specificity.

TABLE 3
Deep-sequencing analysis of SaCas9 variants T392A
and N413A/N419A/R654A for on-target and off-target
of VEGFA_8 gRNA. (The % of indel and % of dsODN
integration at each site are listed, % indel/% dsODN integration)

| | | | | N413A/ |
|---|---|---|---|---|
| SaCas9 | wt | T392A | wt | N419A/R654A |
| On-target | 71.02/7.12 | 50.25/7.68 | 64.6/14.57 | 48.9/12.12 |
| OT1 | 0.65/0.1 | 0.44/0.06 | 0.06/0.06 | 0.09/0 |
| OT3 | 0.48/0.06 | 0.28/0.05 | 0.28/0.19 | 0/0 |
| OT4 | 0/0 | 0/0 | 0/0 | 0/0 |
| OT6 | 0/0 | 0/0 | 0/0 | 0/0 |
| OT7 | 0.11/0.05 | 0/0 | 0/0 | 0/0 |
| OT14 | 0/0 | 0/0 | 0/0 | 0/0 |
| OT15 | 0/0 | 0/0 | 0/0 | 0/0 |
| OT16 | 0/0 | 0/0 | 0/0 | 0/0 |
| OT17 | 0.06/0 | 0/0 | 0/0 | 0/0 |
| OT20 | 0/0 | 0/0 | 0/0 | 0/0 |
| OT21 | 0.85/0.41 | 0/0 | 0.44/0.22 | 0/0 |

### Example 4

This example illustrates the generation of SaCas9 nuclease variant with mutations that has improved specificity, wherein the variant combines the different mutations selected from the claims.

As shown in Table 4, SaCas9 variants with combination mutations at N44A/T316Y, R61A/T316Y, T316Y/T392A, T316Y/K482W, K482W/T392A and N413A/N419A/R654A/T316Y demonstrated an improved specificity compared to wild type SaCas9.

TABLE 4
Deep-sequencing analysis of SaCas9 variants N44A/T316Y, R61A/T316Y, T316Y/T392A, T316Y/K482W,
K482W/T392A and N413A/N419A/R654A/T316Y for on-target and off-target of VEGFA_8 gRNA. (The
% of indel and % of dsODN integration at each site are listed, % indel/% dsODN integration)

| | | | | | | N₄₁₃A/N₄₁₉A/ | |
|---|---|---|---|---|---|---|---|
| SaCas9 | wt | N₄₄A/T₃₁₆Y | R₆₁A/T₃₁₆Y | T₃₁₆Y/T₃₉₂A | T₃₁₆Y/K₄₈₂W | R₆₅₄A/T₃₁₆Y | K₄₈₂W/T₃₉₂A |
| On-target | 72.54/3.59 | 65.01/3.80 | 50.76/3.29 | 63.00/4.88 | 39.00/2.68 | 48.81/4.17 | 36.54/2.27 |
| OT1 | 0.06/0 | 0/0 | 0/0 | 0/0 | 0/0 | 0/0 | 0/0 |
| OT2 | 0.16/0 | 0.11/0 | 0/0 | 0.08/0 | 0/0 | 0/0 | 0/0 |
| OT3 | 0.12/0 | 0.12/0 | 0/0 | 0/0 | 0/0 | 0/0 | 0.15/0 |
| OT4 | 0/0 | 0/0 | 0/0 | 0/0 | 0/0 | 0/0 | 0/0 |
| OT7 | 0/0 | 0/0 | 0/0 | 0/0 | 0/0 | 0/0 | 0/0 |
| OT10 | 0/0 | 0/0 | 0/0 | 0/0 | 0/0 | 0/0 | 0/0 |
| OT13 | 0.00/0 | 0/0 | 0/0 | 0/0 | 0/0 | 0/0 | 0/0 |
| OT16 | 0/0 | 0/0 | 0/0 | 0/0 | 0/0 | 0/0 | 0/0 |
| OT17 | 0/0 | 0/0 | 0/0 | 0/0 | 0/0 | 0/0 | 0/0 |
| OT18 | 1.2/0 | 0/0 | 0/0 | 0/0 | 0/0 | 0/0 | 0/0 |
| OT21 | 0.19/0 | 0/0 | 0/0 | 0/0 | 0/0 | 0/0 | 0/0 |

1. A polypeptide comprising a variant of Staphylococcus aureus Cas9 (SaCas9) nuclease, wherein the variant comprises at least 70% identity to SEQ ID NO: 1 and at least one mutation at an amino acid residue of SEQ ID NO: 1 which
(a) is in the vicinity of gRNA nucleotide 12-14;
(b) is in the bridge helix of SaCas9; or
(c) forms a hydrogen bond with a target DNA.

2. The polypeptide of claim 1, wherein the mutation is at the amino acid residue selected from the group consisting of N44, R61, N120, T134, Y230, R245, K248, Y249, T316, S317, G391, T392, N413, N419, I445, L446, S447, K482, Y651, R654, D786, T787, Y789, K815, Y882, R1012, T1019 and S1022.

3. The polypeptide of claim 1, wherein the variant comprises at least one mutation at an amino acid residue of SEQ ID NO: 1 selected from the group consisting of N44, R61, K248, T316, S317, T392, N413, N419, K482, and R654.

4. The polypeptide of claim 1, wherein the mutation is selected from N44A, R61A, T316Y, S317Y, T392A, N413 A, N419A, K482W, and R654A.

5. The polypeptide of claim 1, wherein the mutation is selected from T316Y, S317Y and K482W.

6. The polypeptide of claim 1, wherein the mutation is N44A or R61A,

7. The polypeptide of claim 1, wherein the mutation is T392A or a combination of N413A, N419A and R654A.

8. The polypeptide of claim 1, wherein the mutation is
(a) a combination of N44A and T316Y, or
(b) a combination of R61A and T316Y, or
(c) a combination of T316Y and T392A, or
(d) a combination of T316Y and K482W, or
(e) a combination of K482W and T392A, or
(f) a combination of N413A, N419A, R654A and T316Y.

9. The polynucleotide encoding the polypeptide according to claim 1.

10. A vector comprising the polynucleotide of claim 9.

11. The vector of claim 10, which is a plasmid vector or a viral vector.

12. The vector of claim 11, which is a lentiviral vector, a retroviral vector or an AAV vector.

13. A kit comprising the polypeptide according to claim 1 or a polynucleotide encoding the same, and a guide RNA.

14. The kit of claim 13, further comprising a donor DNA comprising a transgene.

15. A cell comprising a vector for expressing the polypeptide according to claim 1.

16. A method for genome engineering in a cell comprising introducing into the cell an effective amount of the polypeptide according to claim 1 or a polynucleotide encoding the same.

17. The method of claim 16, further comprising introducing into the cell a donor DNA comprising a transgene.

18. The method of claim 16, wherein the cell is a eukaryotic cell.

19. The method of claim 16, wherein the cell is a mammalian or human cell.

20. The method of claim 16, wherein the cell is a one-cell embryo.
